(19) United States
(12) Patent Application Publication
(10) Pub. No.: US 2017/0100520 A1
(43) Pub. Date: Apr. 13, 2017

(54) PATTERNED SURFACE

(71) Applicant: KIMBERLY-CLARK WORLDWIDE, INC., Neenah, WI (US)

(72) Inventor: Russell F. Ross, Atlanta, GA (US)

(21) Appl. No.: 15/316,976

(22) PCT Filed: Jun. 29, 2015
(86) PCT No.: PCT/US2015/038231

§ 371 Date: Dec. 7, 2016

Related U.S. Application Data
(60) Provisional application No. 62/019,105, filed on Jun. 30, 2014.

(51) Int. Cl. A61L 27/50; A61L 27/58; A61L 27/26

(52) U.S. Cl. CPC A61L 27/50; A61L 27/26; A61L 27/58; A61L 2400/18

(57) ABSTRACT

The present disclosure provides patterned materials that may be useful in reducing certain negative effects associated with damaged tissue in vivo. The patterned materials can modify the healing process, and may minimize the formation of scar tissue. Such effects can provide inhibition of adhesion between tissues and/or reduction of fibrotic encapsulation around implanted medical devices.

### CROSS-REFERENCE TO RELATED APPLICATION

This application claims the benefit of U.S. Provisional Patent Application 62/019,105, which was filed on Jun. 30, 2014, the entire contents of which are incorporated herein by reference.

### FIELD OF THE DISCLOSURE

The present subject matter relates generally to patterned materials that may be useful in vivo in reducing certain negative biological effects commonly associated with the healing of damaged tissue.

### BACKGROUND

Surgical procedures are widely employed, with over 50 million inpatient surgeries performed yearly. Some of the most common inpatient surgeries include joint replacements, cardiac catheterizations, angioplasties, cesarean sections, and hysterectomies. One common complication associated with certain surgical procedures is the formation of one or more adhesions at or near the site of the surgical procedure. Fibrous tissue (i.e., scar tissue) forms as a natural part of the body's healing process at the site of tissue disturbance. In some cases, such fibrous tissue develops between and connects two surfaces, e.g., two tissue surfaces, including between a tissue surface and an organ surface, forming an adhesion. Adhesions can occur anywhere within the body, although the most common sites are within the abdomen, pelvis, and heart. Although adhesions may be harmless, in some cases adhesions may lead to localized pain, cramping, nausea, limited flexibility and function, pressure, swelling, blockages, and more serious symptoms such as loss of organ function. In addition, adhesions can impair the lifetime of implantable medical devices (e.g., sensors and therapeutic delivery devices).

Abdominal adhesions can occur in up to 93% of patients who undergo abdominal or pelvic surgery. For example, typical abdominal and pelvic adhesions can occur between portions of the small and/or large intestines, liver, gallbladder, uterus, ovaries, fallopian tubes, and bladder. In some cases, abdominal adhesions can constrain the normal movement of the small or large intestines, pulling or twisting them out of place, which can lead to intestinal obstruction. Pelvic adhesions can lead to infertility, repeated miscarriages, and increased incidence of ectopic pregnancy. Cardiac adhesions are a relatively common complication encountered following open heart surgery. After virtually every open heart procedure, extensive adhesions form (e.g., between a surface of the heart and the inner surface of the sternum). Such adhesions can lead to restricted heart function. All types of adhesions may require additional surgery to treat the adhesions, which may, in some cases, lead to the development of further adhesions.

Prevention and/or reduction of adhesions is not straightforward; however, various strategies have been studied and/or developed for limiting the incidences of adhesions. Anti-adhesion adjuvants applied to the site of the surgical procedure can decrease the formation of adhesions by providing a mechanical barrier between affected tissues, preventing their adhesion. For example, fluid barriers or surgical membranes comprising such materials as polysaccharides (e.g., cellulose and/or hyaluronic acids) can be employed to prevent adhesions in the specific area of application. Another strategy for the prevention of adhesions is the application of one or more local therapeutics, including but not limited to, anticoagulants, fibrinolytics, and anti-inflammatories (e.g., NSAIDs, prostaglandins, and antihistamines). There are mixed results regarding effectiveness of such approaches and no one approach has proven to be ideal for inhibiting adhesions in all surgeries.

It would be useful to provide other materials and methods that can effectively decrease the severity and/or incidences of adhesions, which may lead to a reduction in the need for further surgeries and/or an improvement in long-term medical implant function.

### SUMMARY

An aspect of this disclosure relates to the provision and use of a material having at least one patterned surface. The specific types of patterning described herein can, in some embodiments, be beneficial in treating wounds, e.g., through modifying the healing of damaged tissues. In some embodiments the materials described herein are intended for use as adjuncts in vivo to reduce the development of scar tissue (e.g., to reduce the incidence, extent, and/or severity of post-operative adhesions). This effect may be achieved via biological mechanisms rather than simply by mechanical means. In certain embodiments, it is believed that such materials can specifically impact cellular responses by modulating gene expression.

In one aspect of the present disclosure, a patterned adhesion barrier comprising a base surface is provided, wherein at least a portion of the base surface comprises a plurality of raised structures (e.g., projection) attached to the base surface and extending outward therefrom, wherein the raised structures (e.g., projections) are irregularly spaced with respect to each other and have an average length to diameter aspect ratio of at least about 5. In certain embodiments, the average length to diameter aspect ratio is higher, e.g., at least about 10 or at least about 15. In some embodiments, all raised structures (e.g., projections) or substantially all raised structures (e.g., at least about 90% of the raised structures) within a given region have a length to diameter aspect ratio of at least about 5.

The lengths of the raised structures in the barriers described herein can vary. In certain embodiments, representative average lengths can be at least about 5 μm, at least about 10 μm, or at least about at least about 15 μm. For example, in some embodiments, representative average lengths can be between about 5 μm and about 100 μm, between about 5 μm and about 75 μm, or between about 5 μm and about 50 μm (e.g., between about 15 μm and about 50 μm). In some embodiments, the plurality of projections comprises projections having substantially the same length, wherein lengths vary by less than about 20% with respect to the average length. In other embodiments, the lengths of the raised structures can vary, for example, by at least about 20% with respect to the average length or by at least about 50% with respect to the average length. In some embodiments, the raised structures each have a substantially uniform diameter along their length or can each have a diameter that is highest at the point of attachment to the base surface and decreases along the length of the projection.

In some embodiments, the plurality of raised structures comprises projections having substantially the same diameters (e.g., substantially the same average diameter along the length of the raised structure or substantially the same maximum diameter along the length of the raised structure). The average spacing between adjacent projections can vary. For example, the average spacing between adjacent projections may, in some embodiments, be less than about 1μ. In some embodiments, the spacing between adjacent projections can be related to the average diameter of the projections (e.g., less than about 2 times the average diameter of the projections). The projections can, in some embodiments, be flexible. In some embodiments, the patterned barrier can be flexible. In some embodiments, the plurality of projections define a patterned surface that is not hydrophobic.

The makeup of the patterned adhesion barriers described herein can vary; in some embodiments, the raised structures comprise one or more biocompatible polymers. Exemplary biocompatible polymers include, but are not limited to, polyethylene, polypropylene, poly(tetrafluoroethylene), poly(methyl methacrylate), poly(methacrylic acid), polyethylene-co-vinylacetate, poly(dimethylsiloxane), polyurethane, poly(ethylene terephthalate), polysulfone, poly(ethylene oxide), polyether etherketone, nylon, polyorthoesters, polyanhydrides, polycarbonates, poly(butyric acid), poly(valeric acid), poly(vinyl alcohol), poly(lactic acid), poly(caprolactone), polydioxanone, poly(ortho ester), poly(hydroxy butyrate valerate), poly(glycolic acid), and derivatives and copolymers thereof. In some embodiments, the biocompatible polymers are advantageously bioabsorbable. The patterned adhesion barriers described herein can be associated with a substrate (e.g., a medical device) or can be freestanding.

Methods for use of such materials are also disclosed. For example, in one aspect of the invention, a method for preventing or inhibiting the formation of scar tissue is provided, comprising administering a patterned adhesion barrier as described herein in vivo, adjacent to one or more damaged tissue. The damaged tissue can be, for example, the result of a wound (including a burn) or a surgical procedure. This method can be effectively employed, for example, at surgical sites within the abdominal, pelvic, cardiac, or spinal region. In some embodiments, such a method can prevent or inhibit the formation of adhesions near (including involving) the damaged tissue.

In another aspect, a method for preventing or inhibiting the formation of fibrotic encapsulation of an medical device implanted within a body is provided, comprising administering a patterned barrier as described herein adjacent to the medical device. The administering step can be performed, for example, at the same time as the medical device is implanted or prior or subsequent to the time the medical device is implanted. The patterned barrier can be administered in various forms, including as a freestanding film or in association with the medical device (e.g., as a coating on at least a portion of the device).

In a still further embodiment, a method of decreasing collagen production is provided, comprising administering a patterned material as described herein to one or more cells. In certain embodiments, the decreased collagen production is believed to be associated with a decrease in fibroblast gene expression. Accordingly, in some embodiments, the cells to which the material is administered express fibroblast genes and the patterned material reduces the expression of fibroblast genes in the cell as evidenced by a decrease in the amount of one or more of: TGFβ1 ligand, TβR2 receptor, or Smad3 intercellular mediator in the cell.

For example, in certain embodiments, the projections have an average lengths of at least about 5 μm and the patterned material reduces the expression of fibroblast genes in the cell by at least about 20% as compared with a comparable non-patterned material and in certain embodiments, the projections have an average lengths of at least about 15 μm and the patterned material reduces the expression of fibroblast genes in the cell by at least about 50% as compared with a comparable non-patterned material. In certain embodiments, the projections have an average length to diameter aspect ratio of at least about 5 and the patterned material reduces the expression of fibroblast genes in the cell by at least about 20% as compared with a comparable non-patterned material and in certain embodiments, the projections have an average length to diameter aspect ratio of at least about 15 and the patterned material reduces the expression of fibroblast genes in the cell by at least about 50% as compared with a comparable non-patterned material.

In an additional embodiment, the decreased collagen production is believed to be associated with a modified fibroblast morphology. Accordingly, in some embodiments, the cells to which the material is administered express fibroblast genes and administration of the patterned material leads to changes in fibroblast morphology as compared with fibroblast morphology observed by administering a comparable non-patterned material.

In some embodiments, such changes in fibroblast morphology are evidenced by a reduction in internal cellular tension. In some embodiments, such changes in fibroblast morphology are evidenced by a reduced cell surface area (e.g., wherein the cell surface area is reduced by at least about 50% in comparison to that observed by administering a comparable non-patterned material).

The foregoing presents a simplified summary of some aspects of this disclosure in order to provide a basic understanding. The foregoing summary is not extensive and is not intended to identify key or critical elements of the invention or to delineate the scope of the invention. The purpose of the foregoing summary is to present some concepts of this disclosure in a simplified form as a prelude to the more detailed description that is presented later. For example, other aspects will become apparent from the following.

### BRIEF DESCRIPTION OF THE DRAWINGS

In the following, reference is made to the accompanying drawings, which are not necessarily drawn to scale and may be schematic. The drawings are exemplary only, and should not be construed as limiting the invention.

FIG. 1 is a schematic illustration of a cross-section of a patterned material in accordance with one embodiment of the present disclosure;

FIGS. 2A and 2B are scanning electron microscope (SEM) images of exemplary surface topographies exhibited by certain materials of the present disclosure;

FIG. 3A is a schematic depiction of a lamination method for the preparation of the types of surface topographies described herein, FIGS. 3B and 3C are SEM images of exemplary surface topographies, showing projection geometries, and FIG. 3D is a graph presenting the projection diameter and projection lengths of both long and short projection patterned films;

FIGS. 4A, 4B, and 4C are graphs depicting the effect of raised structure (projection) length on the growth of myofibroblast-specific genes, where ** indicates p<0.01;

FIGS. 5A, 5B, and 5C are graphs depicting the effect of raised structure (projection) length on the expression and activation of TGFβ pathway genes, where * indicates p<0.05 and ** indicates p<0.05;

FIG. 5D provides images of fibroblasts in the presence of flat, short, and long projections;

FIGS. 6A-6C are SEM images of 3T3 fibroblasts on flat, short, and long projections; FIGS. 6D-6F are SEM images of 3T3 fibroblasts on flat, short, and long projections, indicating cellular attachments (white arrows);

FIGS. 7A-7C are images of 3T3 fibroblasts stained with rhodamine phalloidin for F-actin on flat, short, and long projections; FIGS. 7D-7F are images of 3T3 fibroblasts stained for pMLC on flat, short, and long projections; and

FIG. 8A is a schematic illustration of a mouse model indicating the positioning of flat (F) and long (M) patterned films (comprising projections as described herein) inserted subcutaneously in the dorsal aspect of wild type mice; FIG. 8B provides images of trichrome stained histological sections for these two regions; FIG. 8C provides higher magnification images of FIG. 8B; FIG. 8D provides images of such sections immunohistologically stained for collagen I and III (wherein the area surrounding the implanted film is indicated as a white dashed line); and FIG. 8E provides higher magnification images of FIG. 8D.

### DETAILED DESCRIPTION

Exemplary embodiments are described below and illustrated in the accompanying drawings, in which like numerals refer to like parts throughout the several views. The embodiments described provide examples and should not be interpreted as limiting the scope of the inventions. Other embodiments, and modifications and improvements of the described embodiments, will occur to those skilled in the art, and all such other embodiments, modification, and improvements are within the scope of the present invention.

In general, the present disclosure provides materials having a base surface with raised structures thereon (i.e., producing patterned or textured surfaces), designed for in vivo use. Although the materials can act as mechanical barriers between internal tissues and organs, they are advantageously capable of decreasing collagen production, which is believed to occur via altering gene expression. The raised structures on the base surface can define unique surface topographies (e.g., nanotopographies and/or microtopographies), capable of influencing one or more cellular pathways when the disclosed materials are brought into contact with a cellular environment (e.g., within a surgical site).

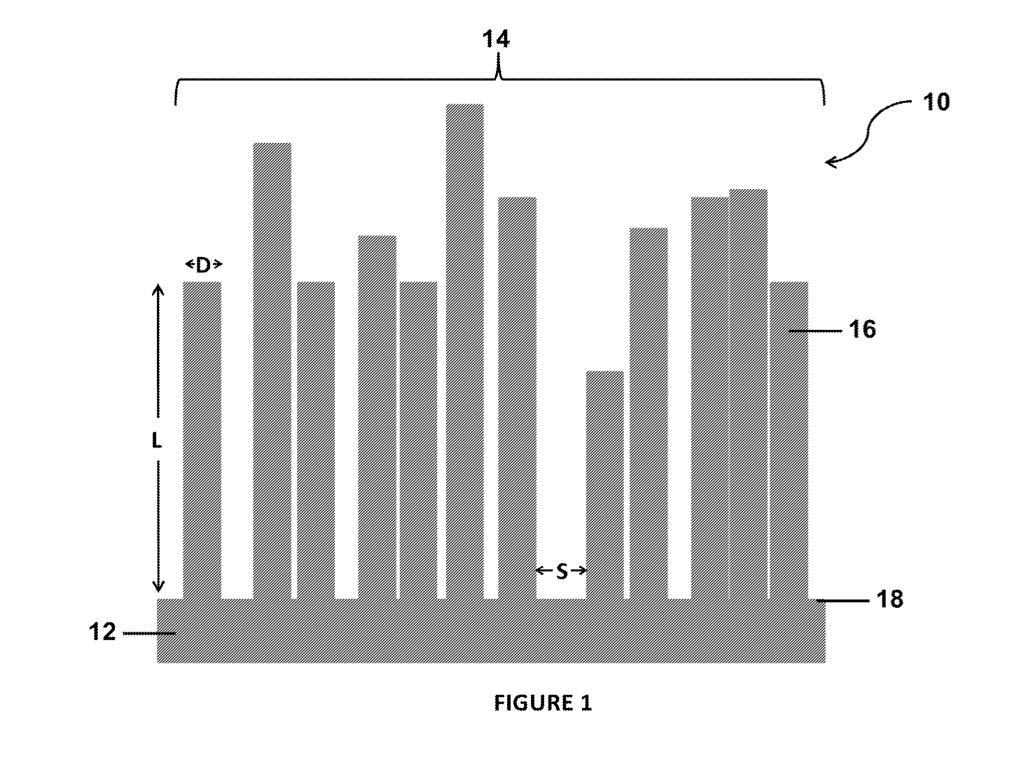

The raised structures defining the patterned surfaces described herein can vary, for example, in shape, size, and spatial arrangement on the surface (e.g., density and regularity). A schematic drawing of a material cross-section of an exemplary embodiment of the present disclosure is provided in FIG. 1. FIG. 1 shows a patterned material 10, comprising a base 12 having a base surface 18 to which a plurality of raised structures 16 are attached. Relevant dimensions of each raised structure 16 include the length, L, the cross-sectional diameter D, and the inter-structure spacing S of the raised structures. The raised structures 16 provide a patterned surface 14.

The raised structures 16 can comprise a plurality of identical structures or may include different structures of various sizes, shapes, and combinations thereof. Exemplary shapes of raised structures include, but are not limited to, fibers, tubes, cones, ridges, hills, plateaus, cubes, spheres, and the like. In some embodiments, the raised structures comprise “fibers,” which can be alternatively referred to as “posts,” “columns,” or “pillars.” In the projections described herein, the length L of each projection is typically greater than the average diameter D of that projection. Exemplary projections are illustrated in FIG. 1 and can be described as elongated structures extending lengthwise from the surface to which they are attached. Projections commonly have substantially cylindrical shapes. In some embodiments, the diameter of a projection is relatively consistent along the length L, whereas in other embodiments, the diameter of a projection can vary along the length (e.g., with a large diameter at the base of the projection at the surface to which it is attached, with a tapered shape leading to a smaller diameter at the top of the projection). Where the diameter of the projection varies along its length, the diameter D referred to herein is intended to refer to the maximum cross-sectional diameter of the projections.

Although the remainder of the disclosure is described with respect to raised structures comprising projections, it is noted that this disclosure is not intended to preclude the use of other raised structure shapes in place of or in addition to such projections. It is to be understood that the dimensions of other raised structure shapes can be modified within the ranges described herein and based on the disclosure specific to projections presented herein.

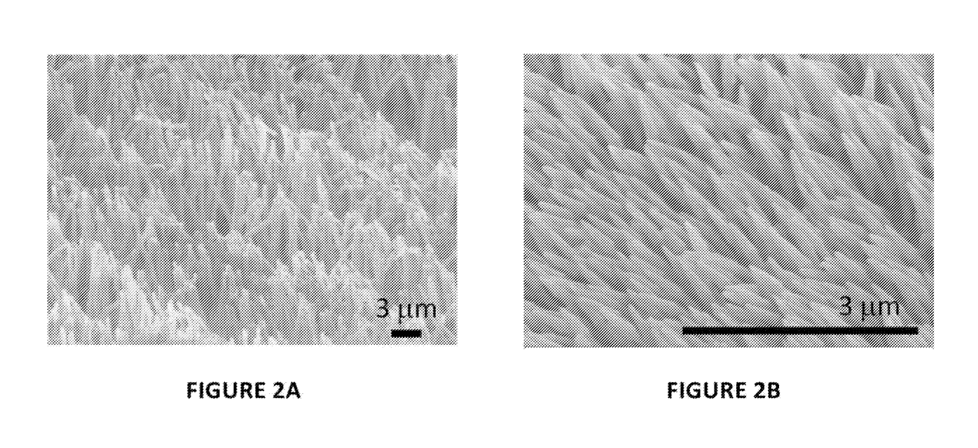

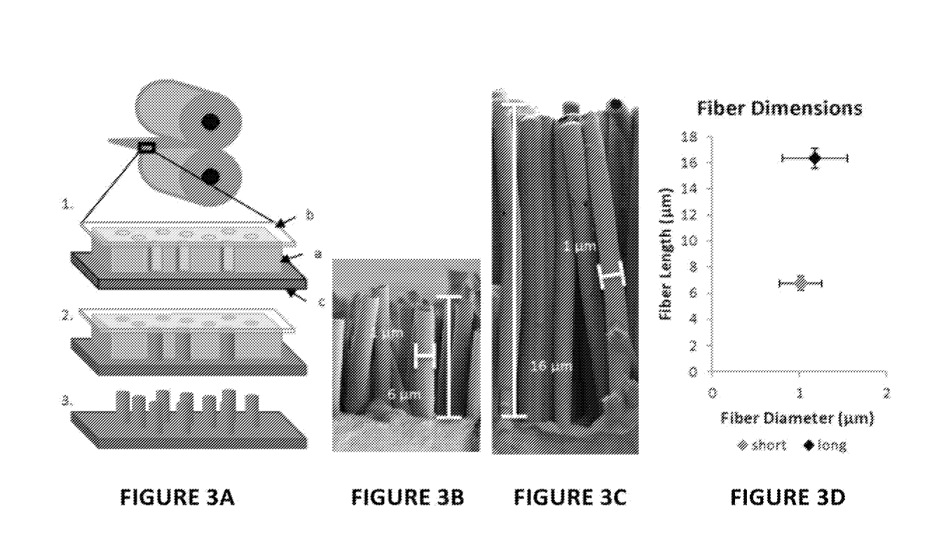

Representative dimensions of the raised structures described herein can be, for example, between about 1 nm and about 100 nm and/or between about 100 nm (0.1 μm) and about 100 μm. Although not intended to be limiting, certain such raised structures can be projections having diameters D ranging from about 10 nm to about 10 μm, e.g., from about 0.1 μm to about 5 μm or from about 0.5 μm to about 2 μm. As shown in FIGS. 2A and 2B, certain embodiments comprise projections having average diameters of less than 1 μm (e.g., between about 10 nm and about 1 μm). As shown in FIGS. 3B and 3C, certain embodiments comprise projections having average diameters of about 1 μm. In certain patterned material according to the present disclosure, the raised structures can have substantially the same diameter or the raised structures can comprise a plurality of structures having two or more different diameters.

Projection lengths L can be widely variable, but are typically in the microscale range. In various embodiments, certain projections can have lengths from base to tip of at least about 0.1 μm, at least about 0.5 μm, at least about 1 μm, at least about 3 μm, at least about 5 μm, at least about 10 μm, or at least about 15 μm. In some embodiments, projections can have lengths from base to tip of between about 0.1 μm and about 100 μm, such as between about 1 μm and about 100 μm, between about 5 μm and about 75 μm, or between about 5 μm and about 50 μm (e.g., between about 15 μm and about 50 μm). For example, as shown in FIGS. 3B and 3C, certain embodiments comprise projections having lengths of between about 5 μm and about 20 μm. Specifically, the data presented herein refers to “short” projections having lengths of about 6 μm and “long” projections having lengths of about 16 μm (with some variance, as shown in FIG. 3D). Although not intended to be limiting, in some embodiments, “longer” projections (e.g., those having lengths of at least about 10 μm, at least about 12 μm, or at least about 14 μm) exhibit particularly advantageous biological effects. It is noted that these values may be dependent, in part, on projection diameter, with smaller diameter projections requiring smaller lengths to achieve similar results (a detailed discussion of length:diameter aspect ratio is provided below).

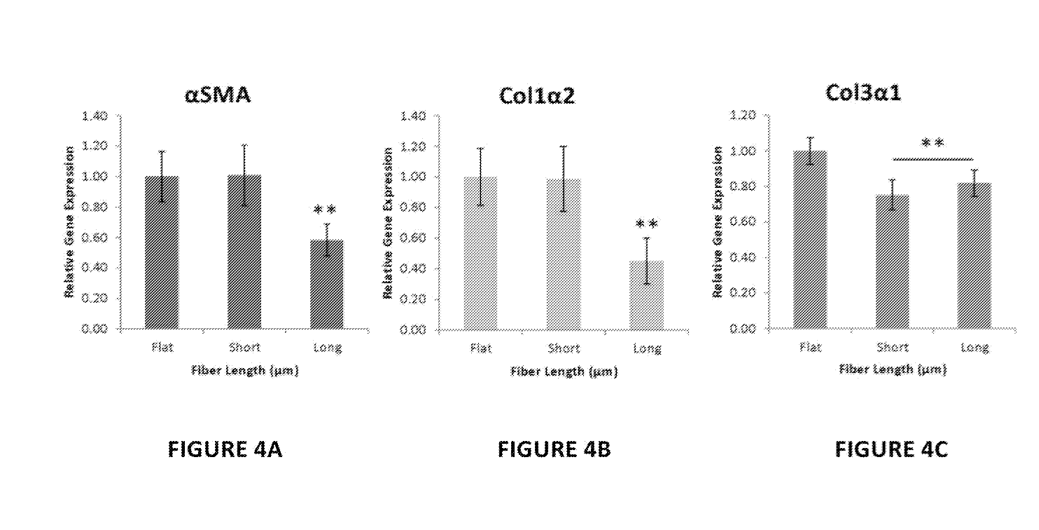

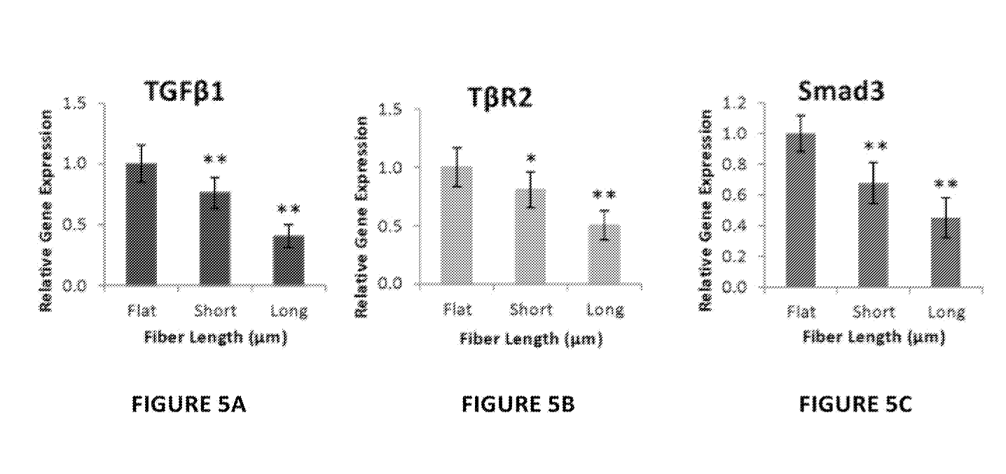

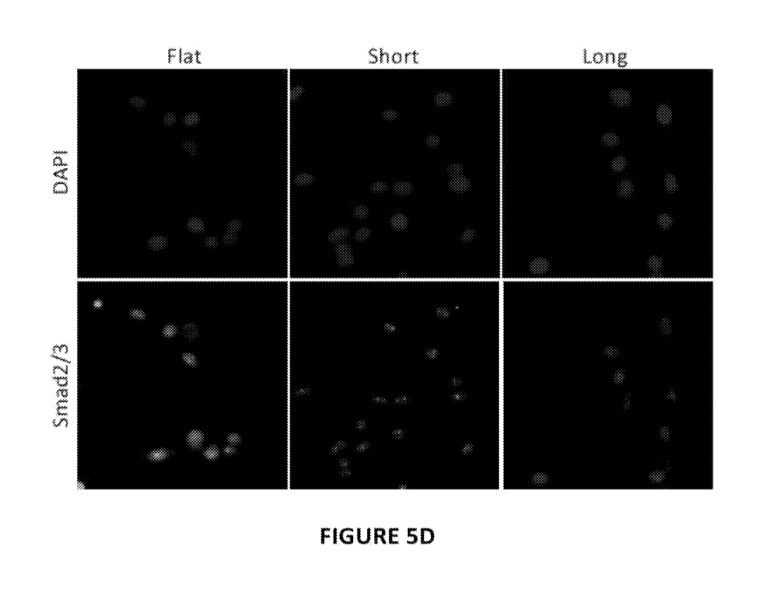

The data presented in FIGS. 4A, 4B, and 4C demonstrates that fibroblast-specific gene expression (particularly expression of αSMA and Collα2 myofibroblast specific genes, as shown in FIGS. 4A and 4B) advantageously decreases with increasing projection lengths (with projection diameter remaining constant). Furthermore, the data presented in FIGS. 5A, 5B, and 5C demonstrates that as projection length increases (with projection diameter remaining constant), TGF-β pathway gene expression and activation decreases. Specifically, FIG. 5A provides expression data for the TGFβ1 ligand, FIG. 5B provides expression data for the receptor TβR11, and FIG. 5C provides expression data for the intercellular mediator Smad3.

In certain embodiments, the projections within a given patterned region comprise projections of different lengths L. For example, in some embodiments, a patterned region comprises two types projections (each type having a different length L), which can each be in designated areas within the patterned region(s) or can be dispersed (e.g., randomly). The patterned region(s) can comprise even higher numbers of projection types (each type having a different length L). For example, as shown in the schematic of FIG. 1, some patterned regions can comprise projections of multiple different lengths, randomly spatially dispersed across the base surface 18.

The range of different lengths of the projections can vary within a patterned region. This range can be described, for example, by variance from the average length within the region. For example, in some embodiments, the majority of projections can be described has having substantially the same length (e.g., wherein the lengths vary by less than about 10% with respect to the average length, less than about 20% with respect to the average length, or less than about 30% with respect to the average length). In other embodiments, the majority of projections can be described as having different lengths (e.g., wherein the lengths vary by at least about 20% with respect to the average length, at least about 30% with respect to the average length, at least about 50% with respect to the average length, at least about 70% with respect to the average length, or at least about 90% with respect to the average length). In certain such embodiments, at least about 90%, at least about 95%, at least about 98%, or at least about 99% of the projections fall within these ranges.

Projection lengths that are particularly useful with regard to the materials described herein are dependent on projection diameters. In other words, projections can, in some embodiments, be described in terms of their aspect ratios, i.e., the ratio of projection length to projection diameter. Exemplary aspect ratios of projections that are useful in regard to the present disclosure include aspect ratios of at least about 5:1. It is noted that particularly beneficial biological results are observed when the projections have an aspect ratio of at least about 5:1. In some embodiments, the aspect ratios are even higher, e.g., at least about 6:1, at least about 8:1, at least about 10:1, at least about 12:1, or at least about 15:1. Exemplary average aspect ratio ranges are between about 5:1 and about 50:1, between about 5:1 and about 25:1, between about 10:1 and about 50:1, and between about 10:1 and about 25:1. Preferably, all or substantially all projections within a given patterned region exhibit such aspect ratios. For example, in some embodiments, each projection has a length to diameter aspect ratio of at least about 5. In some embodiments, at least about 75%, at least about 80%, at least about 85%, at least about 90%, at least about 95%, at least about 98%, or at least about 99% of the projections in a given patterned region exhibit such aspect ratios (e.g., an aspect ratio of at least about 5).

In certain embodiments, the lengths and aspect ratios of the projections are such that the patterned surface exhibits some degree of “flexibility.” As reflected in the images of FIGS. 2A and 2B, the distal ends of the projections are advantageously capable of some degree of movement. In some embodiments, the distal ends of the projections can touch and/or can interact with one or more other projections, e.g., causing clumping, as seen in the images of FIGS. 2A and 2B. In some embodiments, the flexibility can be defined by the shear modulus of the material. For example, in certain embodiments, the shear modulus is less than about 400 mPa. Desirable ranges include a shear modulus within the range of about 10 mPa to about 200 mPa, e.g., about 10 mPa to about 100 mPa or about 20 mPa to about 200 mPa or about 20 mPa to about 100 mPa, including about 20 mPa to about 50 mPa.

The variance in length between the projections within a given patterned region can, in some embodiments, be quantified by the “roughness” of the patterned surface 14. Methods for determining surface roughness are generally known in the art. For instance, an atomic force microscope in contact or non-contact mode may be utilized according to standard practice to determine the surface roughness of a material. Surface roughness that may be utilized to characterize the raised structures on the patterned surface may include the average roughness (R_{A}), the root mean square roughness, the skewness, and/or the kurtosis. Roughness values for the materials described herein are dependent, in part, on projection lengths. However, in general, the average surface roughness (i.e., the arithmetical mean height of the surface roughness parameter as defined in the ISO 25178 series) of exemplary materials described herein, defining the topography thereon, may be within the range of about 50 nm to about 2000 nm (e.g., 75 nm to about 1500 nm) based on root mean square roughness.

Advantageously, the presently disclosed materials comprise at least one region having a high density of raised structures. As demonstrated by the embodiments shown in FIGS. 2A, 2B, 3B, and 3C, in certain embodiments, it is advantageous to provide the raised structures in a closely packed (high density) arrangement with respect to each other. Inter-structure spacings (shown as “S” in FIG. 1) refer to the shortest lateral dimension of the available space/gap between adjacent raised structures. The average inter-structure spacings described herein are measured at the base of the projection (i.e., at the point of attachment to the base surface), and describe the shortest lateral dimension of the available space/gap between adjacent raised structures. It is understood that the spacings are 2-dimensional and that a given projection may have one spacing value with respect to one adjacent projection and a second (different) spacing value with respect to another adjacent projection.

Relevant inter-structure spacings S can be dependent on projection diameters D, as larger inter-structure spacings may be employed for projections having larger diameters. In certain embodiments, the inter-structure spacing S is, on average, less than about 5 times, less than about 2 times, or less than about 1 times the average diameter D of the raised structures. In some embodiments, adjacent raised structures can be touching. In certain embodiments, in at least a region of the patterned surface, some raised structures can be described as exhibiting close packing/hexagonal packing with respect to one another. The packing can be described in terms of filled area (comprising projections) divided by total area of a region. Such values can range, in various embodiments of the present disclosure, including values of less than or equal to about 0.76 (which represents close packing, assuming the base of each projection is circular in shape; this values may deviate somewhat where the bases of projections deviate from a circular shape). Representative inter-structure spacings (from the base of one raised structure to the base of an adjacent raised structure) can be, for example, less than about 10 μm, less than about 5 μm, less than about 2 μm, less than about 1 μm, less than about 0.5 μm, or less than about 0.1 μm (e.g., between about 10 nm and about 1 μm).

The patterned surfaces of the present disclosure can comprise a non-random pattern (e.g., an organized array) or a random pattern of such raised structures on the. Accordingly, the patterned surfaces can comprise a narrow range of inter-structure spacings S (e.g., where all raised structures are equidistant from one another) or a wide range of inter-structure spacings. Particularly advantageous according to the present disclosure are random patterns of raised structures, wherein the inter-structure spacings are non-uniform or irregular. By “non-uniform” or “irregular” is meant that the variance from the average inter-structure spacings S within a patterned region of the material is at least about 5%, at least about 10%, at least about 15%, or at least about 20% (e.g., between about 5% and about 100% variance from average).

In particular embodiments, the raised structures are in a high density arrangement having an average inter-structure spacing within the ranges noted above (e.g., between about 50 nm and about 1 μm), where the inter-structure spacing is random. By “random” as used herein is meant that, in some embodiments, two or more different inter-structure spacings are present within a given region of the patterned surface, such that the inter-structure spacings within that region cannot be described by a simple mathematical equation. The randomness or irregularity of inter-structure spacing of a given region can, in some embodiments, be described by its fractal dimension of the pattern.

The fractal dimension is a statistical quantity that gives an indication of how completely a fractal appears to fill space as the recursive iterations continue to smaller and smaller scale. The fractal dimension of a two dimensional structure may be represented as:

$$D = \frac{log N ( s )}{log ( e )}$$

where N(e) is the number of self-similar structures needed to cover the whole object when the object is reduced by 1/e in each spatial direction. Detail regarding the determination of fractal dimensions can be found, for example, in International Application Publication No. WO2013/061209 to Ollerenshaw et al., which is incorporated herein by reference in its entirety. Fractal dimensions typically exhibited by the materials described herein are within the range of 1-2 or 2-3.

In some such embodiments, the raised structures comprise projections of at least two different lengths, e.g., as depicted in FIG. 1. Further, in some such embodiments, the projections are otherwise substantially uniform (i.e., with regard to shape and diameter). The materials described herein can comprise a single patterned region (along with one or more non-patterned regions) or may include multiple regions comprising patterns, which can be the same or different. In some embodiments, the patterned materials comprise patterning on at least one surface, wherein a majority of the at least one surface is patterned as described herein. For example, in certain embodiments, at least about 75%, at least about 80%, at least about 85%, at least about 90%, at least about 95%, at least about 98%, at least about 99% of the surface is patterned as described herein. The patterning can comprise a continuous pattern (wherein the given percentage of the surface that is patterned is continuous) or can comprise a discontinuous pattern (with gaps between patterned regions), e.g., in the form of a checkerboard-type or other larger scale regular or irregular pattern.

The overall sizes and shapes of the patterned materials disclosed herein can vary widely and may be tailored with regard to the particular application. In some embodiments, the materials can be produced as large scale films, and cut into individual patch-type units; in other embodiments, such patch-type units can be directly produced. The dimensions of the materials disclosed herein are typically at least as large as the area which the materials are designed to interact with (e.g., at least as large as the damaged tissue site to be addressed). For example, the materials can range from a size comparable to that of the damaged tissue up to a size roughly two or three times larger than the damaged tissue.

In some embodiments, the materials are provided with dimensions of about 1 mm×about 1 mm to about 40 cm×about 40 cm (e.g., between about 10 mm×about 10 mm to about 20 cm×20 cm or between about 1 cm×1 cm and about 10 cm×10 cm). Of course, it is to be understood that such units are not always in square form, and this disclosure is not limited to materials exhibiting equal length and width dimensions. Other shapes, generally consistent with the sizes described herein are also intended to be encompassed within the present disclosure. Accordingly, the materials can be described in terms of area, e.g., as having areas of between about 1 square mm and about 1600 square cm, e.g., between about 100 square mm and about 400 square cm or between about 1 square cm and about 100 square cm.

The thickness of the materials disclosed herein can also vary. In certain embodiments, the thickness of the base 12 can be within the range of about 1-15 microns or more. Typically, longer projections require a thicker base, whereas a thinner base can be used with shorter projections. Of course, where such materials are used in combination with a substrate, the overall thickness of the material (including the patterned material and the substrate) can be greater, taking into account the thickness of the patterned material as well as the thickness of the patterned substrate.

The composition of the patterned materials described herein can vary. Advantageously, in preferred embodiments, the materials are nontoxic and easily sterilized, rendering them suitable for use in vivo. In preferred embodiments, the composition of the base surface 18 on which the raised structures are arranged is the same as the composition of the raised structures 16 themselves, although the disclosure also encompasses materials wherein the composition of the base surface on which the raised structures are arranged is different from that of the raised structures. Such compositions include metals, ceramics, semiconductors, organics, polymers, etc., as well as composites thereof. By way of example, pharmaceutical grade stainless steel, titanium, nickel, iron, gold, tin, chromium, copper, alloys of these or other metals, silicon, silicon dioxide, and/or polymers may be utilized in forming the materials described herein.

In some embodiments, one or both of the base surface 18 and the raised structures 16 comprise a biocompatible polymer. The term “biocompatible” generally refers to a composition that does not substantially adversely affect the cells or tissues in the area where the material is to be provided (e.g., within a surgical site). It is also intended that the materials do not cause any substantially medically undesirable effect in any other areas of a living subject in which the material is provided. Biocompatible materials may be synthetic or natural. Biocompatible polymers include, but are not limited to, natural polymers (e.g., polysaccharides such as starch, cellulose, and chitosan) and synthetic polymers (e.g., polyethylene (PE), polypropylene (PP), poly(tetrafluoroethylene) (PTFE), poly(methyl methacrylate) (PMMA), poly(methacrylic acid) (PMA), polyethylene-co-vinylacetate (EVA), poly(dimethylsiloxane) (PDMS), polyurethane (PU), poly(ethylene terephthalate) (PET), polysulfone, poly(ethylene oxide) (PEO/PEG), polyether etherketone (PEEK), nylon, polyorthoesters, polyanhydrides, polycarbonates (e.g., tri-methylene carbonate (TMC)), poly(butyric acid), poly(valeric acid), poly(vinyl alcohol) (PVA), poly(lactic acid) (PLA), poly(caprolactone) (PCL), polydioxanone (PDS), poly(ortho ester) (POE), poly(hydroxy butyrate valerate) (PHBV), poly(glycolic acid) (PGA), and derivatives and copolymers thereof (e.g., poly(lactide-co-glycolide), poly(lactide-co-caprolactone)).

In some embodiments, one or more of the polymers used to produce the materials of the present disclosure are bioabsorbable within a reasonable period of time. Representative bioabsorbable materials include, but are not limited to, PGA, PLA, POE, PCL, PHBV, TMC, PLA-co-PGA, PLA-co-PCL, and the like. Bioabsorbable materials can be selected and/or tailored (e.g., by providing mixtures of polymers, copolymers, or derivatives) to allow for complete absorption of the patterned material within any desired timeframe (e.g., between about 1 day and a few months following introduction of the material within a surgical site).

Specific results described herein with regard to the biological effects of patterned surfaces have been observed regardless of the chemical composition of the surface; accordingly, it is believed that a wide range of compositions can be effectively employed to prepare the materials disclosed herein. Although in some embodiments, the patterned surface 14 is advantageously hydrophobic, the disclosure is not limited to materials comprising hydrophobic or superhydrophobic surfaces. In fact, the materials of the disclosure can, in some embodiments, beneficially exhibit the desirable biological effects described herein without the necessity of using a non-hydrophobic (e.g., hydrophilic) composition to prepare the material and/or applying a hydrophobic coating to the material.

In certain embodiments, one or more therapeutics can be incorporated within, coated on, or otherwise associated with the materials of the present disclosure. For example, where the patterned material described herein is used an adjuvant within a surgical site, one or more therapeutics to be released within the surgical site to promote healing can be used. Exemplary therapeutics include, but are not limited to, anticoagulants, fibrinolytics, and anti-inflammatories (e.g., NSAIDs, prostaglandins, and antihistamines), enzymes, and nucleotide-based therapeutics. Specific therapeutics include, but are not limited to, ibuprofen, dextran, sodium hyaluronate, aprotinin, 5-fluorouracil, antibodies to TFG-β, painkillers, and the like.

The materials described herein can be prepared in a range of sizes and the dimensions of the materials can be suitably adapted to a wide range of applications. The pattern on the surface thereof can, in some embodiments, extend over an entire surface of the film, or may be provided only in discrete sections of the film. Furthermore, a pattern can be present on one surface of the film or on two surfaces of the film (wherein the patterns may be the same or different). The thickness of the base 12 and the overall thickness of the material of the embodiments described herein (including the thickness of the base 12 and the length L of the raised structures 16) can be adjusted to an appropriate range for the desired application. In some embodiments, a flexible, drapable, and/or conformable patterned material is provided, which can be readily administered to various sites in vivo.

In some embodiments, the patterned materials described herein can be employed as stand-alone materials. In other embodiments, the patterned materials can be associated with a substrate. A substrate, as used herein, is a physical body onto which a material may be deposited or adhered (e.g., by attaching the base 12 thereto). The patterned materials disclosed herein can be, in some embodiments, associated with various types of substrates, including sheets (backing layers) or other shapes comprising the types of materials noted above, as well as various types of devices. Where a patterned material is associated with a device, it may be advantageous in some embodiments, that at least about 50% of the surface area of the device is covered with the patterned material. For example, about 50% to about 100% of the surface area of the device can be covered, e.g., between about 60% and about 100% or about 70% to about 100%. The coating can be continuous or can be discontinuous. For example, a portion of the surface of the device can be covered with two or more patterned materials as described herein, wherein the materials are the same or different, and wherein they are oriented with respect to one another in a large-scale regular or irregular pattern (e.g., a checkerboard-type pattern). In other embodiments, a large region of the device can be covered with a single patterned material (i.e., in a continuous coated fashion).

The method by which the disclosed patterned materials are produced can vary. For example, in some embodiments, the patterned materials can be prepared according to any standard microfabrication technique including, but not limited to: lithography; etching techniques, such as wet chemical, dry, and photoresist removal (including plasma etching); thermal oxidation of silicon; electroplating and electroless plating; diffusion processes, such as boron, phosphorus, arsenic, and antimony diffusion; ion implantation; film deposition, such as evaporation (filament, electron beam, flash, and shadowing and step coverage), sputtering, chemical vapor deposition (CVD), epitaxy (vapor phase, liquid phase, and molecular beam), electroplating, screen printing, and lamination; stereolithography; laser machining; nanoimprinting, microimprinting, replica molding, and laser ablation (including projection ablation), and growth of structures on the surface.

One exemplary means for the preparation of patterned materials as described herein is by lamination. An exemplary lamination process is depicted in the schematic of FIG. 2A, wherein a microporous polycarbonate membrane (a) is placed between a thin layer of polystyrene (b) and a polypropylene film (c) (Step 1). The layers are then pressed between two rollers at 200° C. and 20 psi, melting the polypropylene film into the microporous membrane (Step 2). Ethylene chloride is then used to etch away the polycarbonate membrane, leaving a patterned film (Step 3). This technique can, in some embodiments, lead to the production of projections having relatively uniform projection diameters and/or uniform projection lengths (see FIGS. 2B, 2C, and 2D). Using this lamination technique, the lengths of the projections can be reproducibly tuned by varying the speed of lamination.

Plasma etching may be utilized, in which deep plasma etching of a material is carried out to create raised structures with diameters on the order of 0.1 μm or larger. Raised structures may be fabricated indirectly by controlling the voltage (as in electrochemical etching). Lithography techniques, including photolithography, e-beam lithography, X-ray lithography, and so forth may be utilized for primary pattern definition and formation of a master die. Replication may then be carried out to form a base surface comprising a plurality of raised structures thereon. Common replication methods include, without limitation, solvent-assisted micromolding and casting, embossing molding, injection molding, and so forth. Self-assembly technologies including phase-separated block copolymer, polymer demixing and colloidal lithography techniques may also be utilized in forming a nanotopography on a surface.

Combinations of methods may be used, as is known. For instance, substrates patterned with colloids may be exposed to reactive ion etching (RIE, also known as dry etching) so as to refine the characteristics of a fabricated raised structure such as diameter, profile, length, pitch, and so forth. Wet etching may also be employed to produce alternative profiles for fabricated raised structures initially formed according to a different process, e.g., polymer de-mixing techniques.

The diameter, shape, and pitch of raised structures may be controlled via selection of appropriate materials and methods. For example, etching of metals initially evaporated onto colloidal-patterned substrates followed by colloidal lift-off generally results in prism-shaped pillars. An etching process may then be utilized to complete the structures as desired. Ordered non-spherical polymeric raised structures may also be fabricated via temperature-controlled sintering techniques, which form a variety of ordered trigonal nanometric features in colloidal interstices following selective dissolution of polymeric nanoparticles. These and other suitable formation processes are generally known in the art (see, e.g., Wood, J. R. Soc. Interface, 2007 Feb. 22; 4(12): 1-17, incorporated herein by reference).

Other methods as may be utilized in forming raised structures, including nanoimprint lithography methods utilizing ultra-high precision laser machining techniques, examples of which have been described in U.S. Pat. No. 6,995,336 to Hunt et al. and U.S. Pat. No. 7,374,864 to Guo et al., both of which are incorporated herein by reference. Nanoimprint lithography is a nanoscale lithography technique in which a hybrid mold is utilized which acts as both a nanoimprint lithography mold and a photolithography mask. Details regarding such a nanoimprint lithography technique are provided in U.S. Patent Application Publication No. 2013/0144257 to Ross et al., which is incorporated herein by reference. The raised structures may also be formed according to chemical addition processes. For instance, film deposition, sputtering, chemical vapor deposition (CVD), epitaxy (vapor phase, liquid phase, and molecular beam), electroplating, and so forth may be utilized for building structures on a surface.

Self-assembled monolayer processes as are known in the art may also be utilized to form the raised structures on the materials disclosed herein. For instance, the ability of block copolymers to self-organize may be used to form a monolayer pattern on the surface. The pattern may then be used as a template for the growth of the desired structures, e.g., colloids, according to the pattern of the monolayer. By way of example, a two-dimensional, cross-linked polymer network may be produced from monomers with two or more reactive sites. Such cross-linked monolayers have been made using self-assembling monolayer (SAM) (e.g., a gold/alkyl thiol system) or Langmuir-Blodgett (LB) monolayer techniques (Ahmed et al., Thin Solid Films 187: 141-153 (1990)) as are known in the art. The monolayer may be crosslinked, which may lead to formation of a more structurally robust monolayer. The monomers used to form the patterned monolayer may incorporate all the structural moieties necessary to affect the desired polymerization technique and/or monolayer formation technique, as well as to influence such properties as overall solubility, dissociation methods, and lithographic methods. A monomer may contain at least one, and more often at least two, reactive functional groups. A molecule used to form an organic monolayer may include any of various organic functional groups interspersed with chains of methylene groups. For instance, a molecule may be a long chain carbon structure containing methylene chains to facilitate packing. The packing between methylene groups may allow weak Van der Waals bonding to occur, enhancing the stability of the monolayer produced and counteracting the entropic penalties associated with forming an ordered phase. In addition, different terminal moieties, such as hydrogen-bonding moieties, may be present at one terminus of the molecules, in order to allow growth of structures on the formed monolayer, in which case the polymerizable chemical moieties may be placed in the middle of the chain or at the opposite terminus. Any suitable molecular recognition chemistry may be used in forming the assembly. For instance, structures may be assembled on a monolayer based on electrostatic interaction, Van der Waals interaction, metal chelation, coordination bonding (i.e., Lewis acid/base interactions), ionic bonding, covalent bonding, or hydrogen bonding.

When utilizing a SAM-based system, an additional molecule may be utilized to form the template. This additional molecule may have appropriate functionality at one of its termini in order to form a SAM. For example, on a gold surface, a terminal thiol may be included. There are a wide variety of organic molecules that may be employed to effect replication. Topochemically polymerizable moieties, such as dienes and diacetylenes, are particularly desirable as the polymerizing components. These may be interspersed with variable lengths of methylene linkers. For an LB monolayer, only one monomer molecule is needed because the molecular recognition moiety may also serve as the polar functional group for LB formation purposes. Lithography may be carried out on a LB monolayer transferred to a substrate, or directly in the trough. For example, an LB monolayer of diacetylene monomers may be patterned by UV exposure through a mask or by electron beam patterning. Monolayer formation may be facilitated by utilizing molecules that undergo a topochemical polymerization in the monolayer phase. By exposing the assembling film to a polymerization catalyst, the film may be grown in situ, and changed from a dynamic molecular assembly to a more robust polymerized assembly.

Any of the techniques known in the art for monolayer patterning may be used. Techniques useful in patterning the monolayer include, but are not limited to, photolithography, e-beam techniques, focused ion-beam techniques, and soft lithography. Various protection schemes such as photoresist may be used for a SAM-based system. Likewise, block copolymer patterns may be formed on gold and selectively etched to form patterns. For a two-component system, patterning may also be achieved with readily available techniques.

Soft lithography techniques may be utilized to pattern the monolayer in which ultraviolet light and a mask may be used for patterning. For instance, an unpatterned base monolayer may be used as a platform for assembly of a UV/particle beam reactive monomer monolayer. The monomer monolayer may then be patterned by UV photolithography, e-beam lithography, or ion beam lithography, even though the base SAM is not patterned. Growth of structures on a patterned monolayer may be achieved by various growth mechanisms, such as through appropriate reduction chemistry of a metal salt and the use of seed or template-mediated nucleation. Using the recognition elements on the monolayer, inorganic growth may be catalyzed at this interface by a variety of methods. For instance, inorganic compounds in the form of colloids bearing the shape of the patterned organic monolayer may be formed. For instance calcium carbonate or silica structures may be templated by various carbonyl functionalities such as carboxylic acids and amides. By controlling the crystal growth conditions, it is possible to control the thickness and crystal morphology of the mineral growth. Titanium dioxide may also be templated.

Other ‘bottom-up’ type growth methods as are known in the art may be utilized, for example a method as described in U.S. Pat. No. 7,189,435 to Tuominen et al., which is incorporated herein by reference, may be utilized. According to this method, a substrate may be coated with a block copolymer film (for example, a block copolymer of methylmethacrylate and styrene), where one component of the copolymer forms nanoscopic cylinders in a matrix of another component of the copolymer. A conducting layer may then be placed on top of the copolymer to form a composite structure. Upon vertical orientation of the composite structure, some of the first component may be removed, for instance by exposure to UV radiation, an electron beam, or ozone, degradation, or the like to form nanoscopic pores in that region of the second component.

In another embodiment, described in U.S. Pat. No. 6,926,953 to Nealey et al., incorporated herein by reference, copolymer structures may be formed by exposing a substrate with an imaging layer thereon, for instance an alkylsiloxane or an octadecyltrichlorosilane self-assembled monolayer, to two or more beams of selected wavelengths to form interference patterns at the imaging layer to change the wettability of the imaging layer in accordance with the interference patterns. A layer of a selected block copolymer, for instance a copolymer of polystyrene and poly(methyl methacrylate) may then be deposited onto the exposed imaging layer and annealed to separate the components of the copolymer in accordance with the pattern of wettability and to replicate the pattern of the imaging layer in the copolymer layer. Stripes or isolated regions of the separated components may thus be formed with periodic dimensions in the range of 100 nanometers or less.

Certain materials of the present disclosure have been shown affect biological processes. For example, in some embodiments, patterned materials as described herein are believed to be capable of affecting cellular function. In certain embodiments, the topographies of the materials defined by the raised structures may be effective in affecting cell signaling, gene replication, gene expression, and/or protein generation. In particular, certain materials disclosed herein can result in a reduced fibrotic response. For example, certain materials described herein may provide a reduction in myofibroblast differentiation via a depression in TGF-β signaling. As such, in some embodiments, the materials of the present disclosure can be effective in diminishing matrix deposition and fibrosis in vivo, rendering them useful in reducing fibrotic encapsulation around implanted medical devices and/or in preventing and/or inhibiting the formation of tissue adhesions.

Certain features that may enhance this biological activity in certain embodiments include: relatively large raised structure length L (e.g., greater than about 10 μm), high raised structure length: diameter aspect ratios (e.g., greater than about 5:1); and/or rough patterned surface, arising from variation in raised structure length. In certain embodiments, materials having one or more of these features is particularly desirable for use according to the disclosed methods.

In certain aspects, a method is provided comprising introduction of a patterned material as described herein adjacent to at least one damaged tissue (including between two tissues, wherein at least one is damaged). For example, the damaged tissue can comprise tissue at the site of a wound, burn, or surgical site. In certain embodiments, the patterned material is introduced into a surgical site (e.g., within a mammalian, such as human body). In certain embodiments, the patterned material is introduced adjacent to (e.g., on at least one surface of, or partially or completely surrounding) an implanted medical device.

In some embodiments, the patterned material in vivo can provide a range of biological effects as described in further detail above and in the Example provided below. In particular, the patterned materials are advantageous in their capabilities of affecting (e.g., reducing/minimizing/decreasing) collagen production and/or normal fibrosis (i.e., scar tissue formation). Consequently, the patterned materials described herein can, in some embodiments, be useful in inhibiting or preventing adhesions between the two tissue surfaces, reducing or preventing the production of external lumps at or near the damaged site (resulting from buildup of scar tissue under the skin), and/or reducing fibrotic encapsulation commonly observed around implanted medical devices.

In some embodiments, the patterned materials shown herein exhibit significantly greater ability to inhibit or prevent adhesions than traditional physical barrier adjuvants that are introduced into surgical sites in a similar manner. Exemplary surgical sites into which the patterned materials described herein are beneficially introduced include, but are not limited to, surgical sites associated with abdominal, gynecological, cardiac, spinal, tendon, peripheral nerve, and thoracic procedures.

### Example

### Patterned Film Fabrication

Patterned films were fabricated by laminating polypropylene films into microporous polycarbonate membranes in a hot roll laminator (Cheminstruments, HL-100), as schematically illustrated in FIG. 3A. Briefly, polystyrene (Sigma, 182427), dissolved in toluene (10% w/v), was spun-coated on to a PET backing layer. The polystyrene was used to cap a microporous polycarbonate membrane (Millipore, ATTP04700), which was then overlaid on pre-pressed polypropylene film (Lab Supply, TF-225-4). All layers were pressed through the hot roll laminator at 20 psi and 210° C. Lamination speed was used to control projection length, with short projections pressed at 0.7 mm/s and long projections at 0.2 mm/s. Polycarbonate and polystyrene were then etched away in two serial washes in methylene chloride for 8 minutes each. All experiments were compared to flat polypropylene film controls processed as above but without the overlaid microporous membrane.

Cell Culture:

Human 3T3 fibroblasts were used for all in vitro studies. Growth media for 3T3 fibroblasts consisted of DMEM high glucose with 10% fetal bovine serum (FBS), 1% sodium pyruvate, and 1% penicillin/streptomycin. Experiments were performed in differentiation media consisting of growth media supplemented with 5 ng/ml TGFβ1 (Peprotech, 100-21).

Scanning Electron Microscope (SEM) Imaging:

To prepare cells adhered to the patterned films for SEM imaging, cells were fixed in 4% paraformaldehyde in PBS for 15 minutes at room temperature, followed by a series of rinses in PBS with increasing concentrations of ethanol. Drying was performed in 100% ethanol with a critical point dryer (Tousimis). Samples of patterned films with and without cells were coated with 10 nm of iridium before imaging in an Carl Zeiss Ultra 55 Field Emission Scanning Electron Microscope using an in-lens SE detector.

Immunofluorescence:

After 48 hours of culture, cells were fixed in 4% paraformaldehyde in PBS for 15 min at room temperature, permeabilized in PBS with 0.5% Triton X-100 for 5 minutes and blocked for 1 hour in 10% goat serum. Primary antibodies were diluted in PBS with 2% goat serum and 3% Triton X-100 and incubated overnight at 4° C. at the following concentrations: Smad2/3 antibody 1:400 (Santa Cruz, sc8332); pMLC 1:50 (Cell Signaling, #3671). Secondary goat anti-rabbit Alexa Fluor 488 (Invitrogen, A11034) was added at a dilution of 1:400 for 1 hour at room temperature. For F-actin staining, rhodamine phalloidin (Invitrogen, R415) was diluted to 1:800 in PBS and incubated with fixed cells for 20 min at room temperature. Nuclei were counterstained in Hoechst dye and cells were visualized using a Nikon Ti-E Microscope. Images were processed in Image J.

QPCR:

RNA was isolated using RNeasy column purification, including an on-column DNase treatment (Qiagen, 74104). The concentration and purity of RNA was determined using a Nanodrop ND-1000 Spectrophotometer (Thermo Scientific).

Approximately 1 μg of RNA was converted to cDNA in a reverse transcription (RT) reaction using the iScript cDNA Synthesis Kit (Bio-Rad, 170-8891). Quantitative PCR analysis of each sample was performed in a ViiA 7 Real Time PCR System (Life Technologies). Forward and reverse intron-spanning primers and Fast SYBR Green Master Mix (Life Technologies, 4385612) were used to amplify each cDNA of interest. Each sample was run in duplicate and all results were normalized to the housekeeping gene L19. Fold changes in gene expression were calculated using the delta-delta Ct method. Figures show the mean and standard deviation for a minimum of 5 biological replicates. For statistical analysis, average expression and standard error of the mean were calculated for each condition across all biological replicates, each of which is an average of two technical replicates. ANOVA analysis followed by Student Newman Keuls test was used to evaluate statistical significance.

In Vivo Studies and Histology:

6 week-old female Swiss-Hamster mice were used for our in vivo studies. Mice were anesthetized with intraperitoneal Avertin. On the dorsal aspect of each mouse, two 0.6 cm incisions were made and a subcutaneous pocket was dissected using surgical microscissors. In the contralateral wounds, each mouse was implanted with one flat control and one patterned film, and then each of the surgical wounds was closed with non-absorbable suture. Two weeks after device placement, the mice were anesthetized, and both dorsal surgical sites were punch excised using a 0.8 cm punch biopsy. Tissue samples were fixed for 24 hours in 4% paraformaldehyde and paraffin embedded. Sections were then either stained with Masson's Trichrome stain, or deparaffinized and immunostained for collagen I and III. For immunostaining, the samples were blocked in 4% BSA, and the following antibodies were used: mouse anti-collagen I at 1:100 dilution (Santa Cruz 80565), goat anti-collagen III at 1:100 dilution (Santa Cruz 8781), anti-mouse Alexa 568 at 1:500 dilution (Invitrogen), anti-goat Alexa 488 at 1:500 dilution (Invitrogen). Images selected for figures are representative of three biological replicates for each treatment group.

### Projection Length Affects Cell Shape and Intercellular Tension.

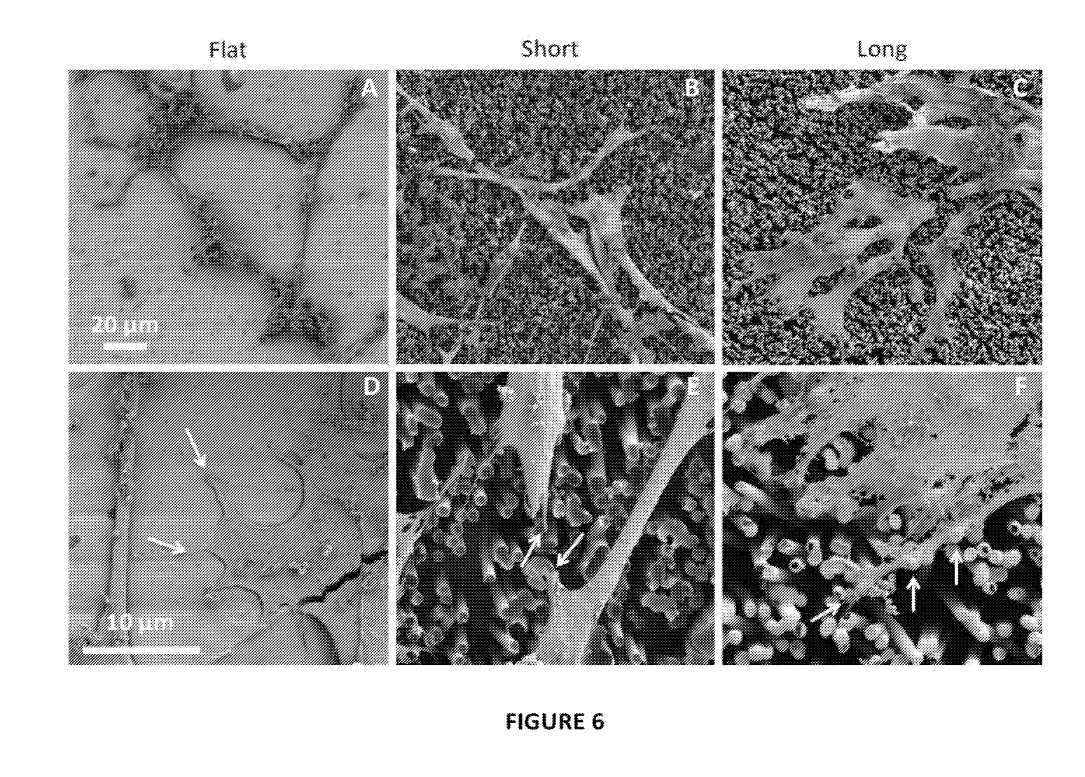

To determine the effect of projection length on fibroblast morphology, two polypropylene films with 1 μm diameter projections of either 6 μm (“short”) or 16 μm (“long”) lengths were fabricated (FIGS. 3B and 3C). 3T3 fibroblasts cultured on both long and short patterned films were imaged by SEM and compared to fibroblasts cultured on flat polypropylene film controls. SEM images reveal a progressive change in fibroblast morphology as projection length increases (FIGS. 6A-C). On flat controls (FIG. 6A), fibroblasts possess elongated cellular projections that emanate from the central cell body and appear to be under tension. On patterned films comprising short projections (FIG. 6B), fibroblasts are also elongated, possessing similar cellular projections. In contrast, on patterned films comprising long projections (FIG. 6C), fibroblasts are devoid of these projections and instead appear much more trapezoidal. Differences in cellular attachment to each film are demonstrated in higher magnification images (FIGS. 6D-6F). On flat controls, fibroblasts form lamellapodia to provide a large area of attachment, while on short projections, cells confine their attachments to a few projections at the ends of cellular projections. In contrast, on long projection films, 3T3 fibroblasts attach to several projections, and these attachments appear to be devoid of cellular tension. The cell body appears draped over the long projections in contrast to the rigid appearance of fibroblasts on the short projection and flat films.

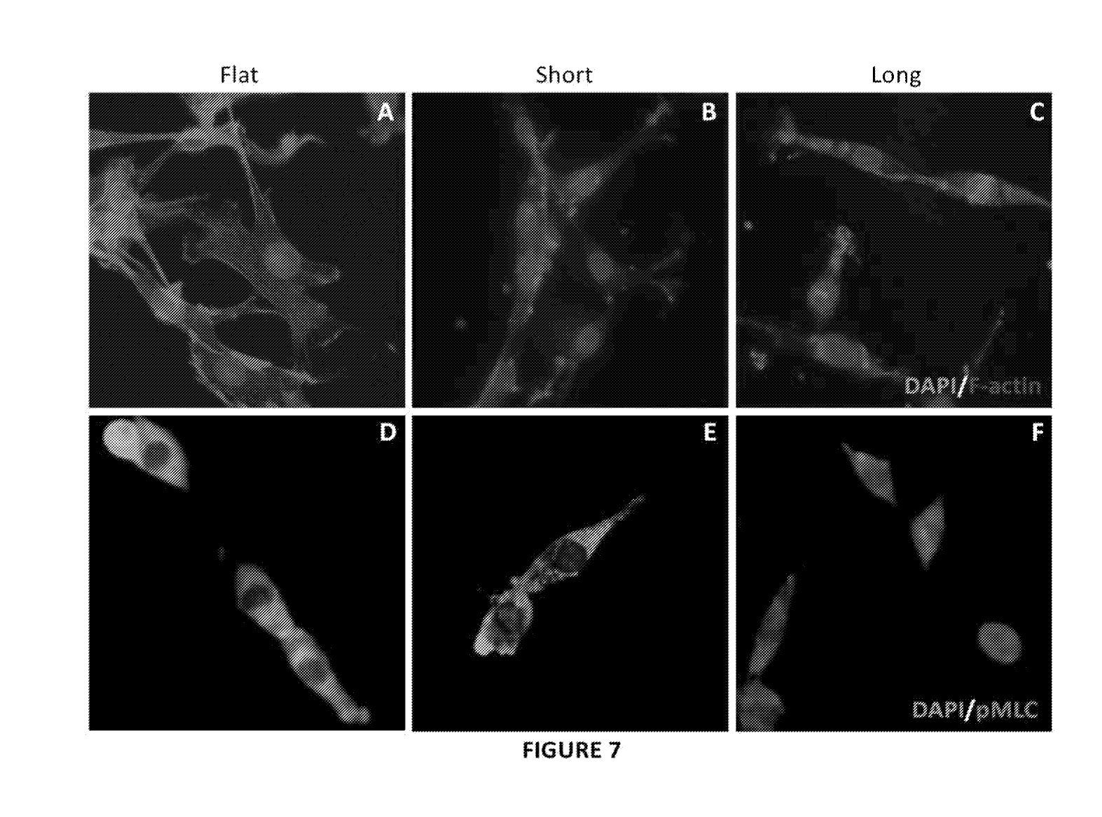

To determine whether the differences in morphology seen in the SEM images correlate with changes in the actin cytoskeleton, cells were stained for F-actin using rhodamine phalloidin (FIGS. 7A-7C). Fibroblasts cultured on flat films form prominent stress fibers with multiple vertices along the cell perimeter, presumably reflecting points of attachment to the substrate. In contrast, fibroblasts grown on either the short or long projection-containing films have less prominent stress fibers, and an overall reduced cell surface area. Quantification of the cell surface area showed a 50% reduction for fibroblasts grown on projections, compared to flat film controls.

Changes in cell morphology and stress fiber formation suggest that culture on such projections may alter intracellular tension generation. Phosphorylation of myosin light chain (pMLC) induces intercellular tension along actin stress fiber; therefore, 3T3 fibroblasts were stained for pMLC after 48 hours of culture (FIGS. 7D-7F). Compared to cells grown on either flat or short projections, pMLC staining in fibroblasts cultured on long projections is diffuse, indicating a decrease in internal cellular tension.

### Long Projections Decrease Myofibroblast Gene Expression and Activation of the TGFβ Pathway.

The morphological and cytoskeletal changes in fibroblasts noted above suggest myofibroblastic differentiation may be decreased in response to long projections. To determine the effect of projection length on myofibroblastic differentiation, 3T3 fibroblasts were cultured on patterned films for 48 hours in the presence of TGFβ1 to induce differentiation toward the myofibroblastic phenotype. While culture on short projection films had no statistically significant effect compared to flat controls, culture on long projections reduced expression of αSMA and Collα2 by 40% and 60%, respectively (FIGS. 4A and 4B). Expression of Col3α1 was marginally reduced on both long and short projection-patterned film, reaching 20% at 48 hours (FIG. 4C). Therefore, projections beyond a certain length seem to effectively reduce myofibroblast-specific gene expression.

As TGFβ directly regulates myofibroblastic gene expression, the effect of projection length on the TGFβ pathway was analyzed. Fibroblasts cultured for 48 hours on either patterned film exhibited a reduction in gene expression of TGFβ signaling components, including TGFβ1 ligand, TGFβ1 receptor 2 (TβRII), and the intercellular mediated Smad (FIGS. 5A-5C). However, consistent with the expression of αSMA and Collα2, knockdown of expression was most pronounced in fibroblasts cultured on long projection-patterned films, with a 50% or greater reduction in all TGFβ signaling genes compared to flat controls. As Smad3 RNA expression was reduced by culture on projection-patterned films, nuclear localization, which indicates activation of Smad2 and 3 by TGFβ, was quantified in 3T3 fibroblasts by immunofluorescence. After 48 hours, the percentage of cells with nuclear localized Smad2/3 does not change significantly between flat films and long and short projections.

However, Smad2/3 staining intensity does appear to change with topography (FIG. 5D). Compared to bright staining on flat films, fibroblasts on short projection patterned films have a clear reduction in staining intensity, with small regions of intensity that may be localized to the nucleoli. On long projection patterned films, nuclear Smad2/3 is the even more diffuse, missing even the small regions of intensity seen on shorter projections. This suggests that, although the fibroblasts appear to be activating Smads in response to TGFβ on all films, there may be a decrease in Smad2/3 protein levels on progressively longer projections which would cause the decrease in staining intensity and possibly explain the reduction in myofibroblastic gene expression.

### Topography Inhibits Surgically-Induced Fibrosis In Vivo

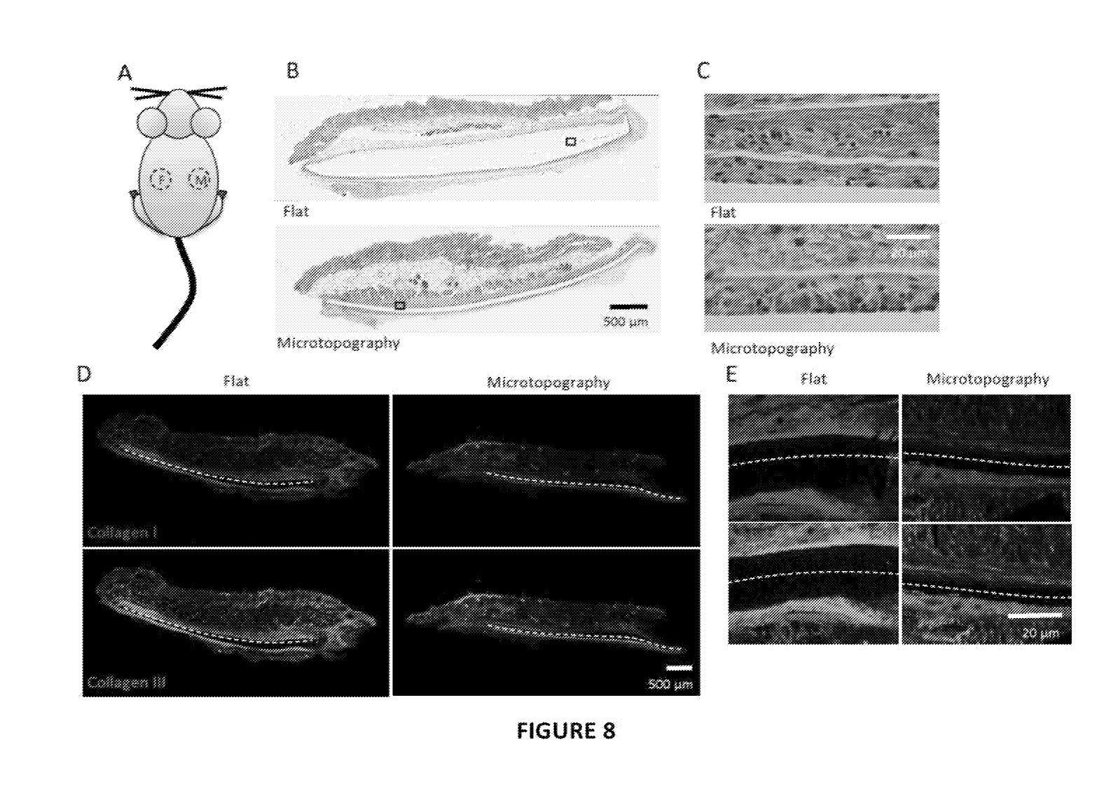

The above experiments in vitro suggest that patterned films reduce myofibroblastic differentiation, and therefore could reduce scar tissue production and encapsulation in vivo. To determine the performance of patterned films in vivo, flat and patterned films were implanted subcutaneously in wild-type adult mice (FIG. 8A). The long projections were chosen for in vivo experiments as they were the films that produced the greatest reduction in myofibroblast activation in vitro. At two weeks post-surgery, histologic analysis with Masson's trichrome stain shows qualitatively sparser deposition of collagen in wounds treated with patterned films (FIG. 8B). Additionally, at high-power magnification, a change in fibroblasts morphology within the wound bed is also observed (FIG. 8C). In wound beds treated with flat films, fibroblasts nuclei adopt an elongated morphology, indicating cell spreading in possible myofibroblast activation. In contrast, fibroblasts grown in wound beds treated with patterned films have nuclei that are more rounded, suggesting a relaxed phenotype. This change in morphology is reminiscent of the morphology of 3T3 fibroblasts seen in vitro via SEM and immunofluorescence.

To identify the expression of specific proteins around in the wound beds, immunohistochemistry was performed. Deposition of both collagen I and III are dramatically reduced in wound beds treated with the patterned films, relative to the flat control films (FIG. 8D). In wound beds treated with flat films, high magnification images demonstrate the greatest staining intensity is located adjacent to the void space containing the inserted film (FIG. 8E). However, wound beds treated with topography have differential staining, such that staining for the collagen I and III is less intense abaxial to the inserted film, compared to the adaxial surface. This orientation is noteworthy because projections are only present on the abaxial side of the inserted film. The opposite side of the patterned film is flat, acting as an internal control, and unsurprisingly, inducing a similar deposition of collagen I and III to that of the flat film treated wound beds.

The above examples are in no way intended to limit the scope of the present invention. It will be understood by those skilled in the art that while the present disclosure has been discussed above with reference to exemplary embodiments, various additions, modifications and changes can be made thereto without departing from the spirit and scope of the invention, some aspects of which are set forth in the following claims.

What is claimed is:

1. A patterned adhesion barrier comprising a base surface, wherein at least a portion of the base surface comprises a plurality of projections attached to the base surface and extending outward therefrom, wherein the projections are irregularly spaced with respect to each other and have an average length to diameter aspect ratio of at least about 5.

2. The patterned adhesion barrier of claim 1, wherein the projections have an average length to diameter aspect ratio of at least about 10.

3. The patterned adhesion barrier of claim 1, wherein the projections have an average length to diameter aspect ratio of at least about 15.

4. The patterned adhesion barrier of claim 1, wherein each projection has a length to diameter aspect ratio of at least about 5.

5. The patterned adhesion barrier of claim 1, wherein the projections have an average length of at least about 5 μm.

6. The patterned adhesion barrier of claim 1, wherein the projections have an average length of at least about 10 μm.

7. The patterned adhesion barrier of claim 1, wherein the projections have an average length of at least about 15 μm.

8. The patterned adhesion barrier of claim 1, wherein the projections each have a substantially uniform diameter along their length.

9. The patterned adhesion barrier of claim 1, wherein the projections each have a diameter that is highest at the point of attachment to the base surface and decreases along the length of the projection.

10. The patterned adhesion barrier of claim 1, wherein the plurality of projections comprises projections having substantially the same length, wherein the lengths vary by less than about 20% with respect to the average length.

11. The adhesion barrier of claim 1, wherein the plurality of projections comprises projections having lengths that vary by at least about 20% with respect to the average length.

12. The adhesion barrier of claim 1, wherein the plurality of projections comprises projections having lengths that vary by at least about 50% with respect to the average length.

13. The patterned adhesion barrier of claim 1, wherein the plurality of projections comprises projections having substantially the same maximum diameters.

14. The patterned adhesion barrier of claim 1, wherein the average spacing between adjacent projections is less than about 1 μm.

15. The patterned adhesion barrier of claim 1, wherein the spacing between adjacent projections is less than about 2 times the average diameter of the projections.

16. The patterned adhesion barrier of claim 1, wherein the projections are flexible.

17. The patterned adhesion barrier of claim 1, wherein the projections comprise one or more biocompatible polymers.

18. The patterned adhesion barrier of claim 17, wherein the one or more biocompatible polymers are selected from the group consisting of polyethylene, polypropylene, poly(tetrafluoroethylene), poly(methyl methacrylate), poly(methacrylic acid), polyethylene-co-vinylacetate, poly(dimethylsiloxane), polyurethane, poly(ethylene terephthalate), polysulfone, poly(ethylene oxide), polyether etherketone, nylon, polyorthoesters, polyanhydrides, polycarbonates, poly(butyric acid), poly(valeric acid), poly(vinyl alcohol), poly(lactic acid), poly(caprolactone), polydioxanone, poly(ortho ester), poly(hydroxy butyrate valerate), poly(glycolic acid), and derivatives and copolymers thereof.

19. The patterned adhesion barrier of claim 17, wherein the biocompatible polymers are bioabsorbable.

20. The patterned adhesion barrier of claim 1, wherein the plurality of projections define a patterned surface that is not hydrophobic.

21. The patterned adhesion barrier of claim 1, wherein the barrier is flexible.

22. The patterned adhesion barrier of claim 1, wherein the barrier is associated with a substrate.

23. The patterned adhesion barrier of claim 22, wherein the substrate comprises an implantable medical device.
